(19) United States
(12) Patent Application Publication
(10) Pub. No.: US 2009/0023822 A1
(43) Pub. Date: Jan. 22, 2009

(54) Method for activating and regenerating catalyst for a fischer-tropsch synthesis reaction

(76) Inventor: Peter J. Tijm, Golden, CO (US)

Correspondence Address: F. LINDSEY SCOTT;LAW OFFICE OF F. LINDSEY SCOTT, 2329 COIT ROAD, SUITE B, PLANO, TX, 75075-3796, US

(21) Appl. No.: 11/879,712

(22) Filed: Jul. 19, 2007

(51) Int. Cl. C07C 1/04; B01J 21/08; B01J 23/75; B01J 38/12

(52) U.S. Cl. 518/715; 502/185; 502/259; 502/303; 502/324; 502/326; 502/335; 502/337; 502/38

(57) ABSTRACT

A system and process to activate, regenerate and use a Fischer-Tropsch catalyst at Fisher-Tropsch vessel reaction temperatures from about 100° C. to about 300° C.

### FIELD OF THE INVENTION

The present invention relates to a process for activating and for regenerating a catalyst containing at least one of cobalt and nickel and optionally a promoter for use in the synthesis of liquid hydrocarbons from carbon monoxide and hydrogen.

### BACKGROUND OF THE INVENTION

Seemingly sustainable increased oil prices have stimulated once again the interest in alternative energy sources. It has brought a renewed interest in the Fischer-Tropsch synthesis as one of the more attractive direct and environmentally acceptable paths to high quality transportation fuels. The Fischer-Tropsch synthesis involves the production of hydrocarbons by the catalyzed reaction of carbon monoxide and hydrogen. Commercial plants have operated in Germany, South Africa, Malaysia and other parts of the world based on the use of particular catalysts. Typically, Fischer-Tropsch catalysts include one or more metals selected from Group VIII of the Periodic Table of Elements (iron, cobalt, nickel, ruthenium, rhenium, palladium, osmium, iridium, platinum), a promoter, and a carrier or support. Cobalt-based catalysts are preferred for the production of a spectrum of hydrocarbons while minimizing the production of carbon dioxide. Nickel-based catalysts tend to produce large quantities of methane; iron-based catalysts produce a spectrum of hydrocarbons, but also generate substantial quantities of carbon dioxide; and ruthenium-based catalysts generate predominantly methane or high melting waxes, depending on the reaction conditions.

Promoters, the function of which we will discuss below, are commonly added to a non-aqueous organic solvent solution or an aqueous solution of a cobalt salt. If desired, non-aqueous organic solvent solutions or aqueous solutions of ruthenium, and/or other promoters like lanthanum, and/or manganese salts, for example, may be prepared and added. Any suitable ruthenium salt, such as ruthenium nitrate, chloride, acetate or the like, or a rhenium salt, such as rhenium nitrate, or the like can be used. In addition, any suitable second promoter metal, e.g., lanthanum salt, such as lanthanum nitrate or lanthanum acetate and/or manganese salt, such as manganese nitrate, or the like can be employed. In general, any metal salt which is either soluble in the organic solvent or aqueous solution of the present invention will not introduce acidity or have a poisonous effect on the catalyst can be utilized.

The use of promoted cobalt containing catalysts is well-known in the art for use in Fischer-Tropsch synthesis. For example, a German commercial operation concentrated on the use of a precipitated cobalt-thoria-kieselguhr fixed-bed catalyst. U.S. Pat. No. 4,088,671 to T. P. Kobylinski (hereafter Kobylinski), which is hereby incorporated in its entirety by reference, describes the use of a ruthenium-promoted cobalt catalyst on a support, such as alumina or kieselguhr, prepared out of a non-aqueous solution of cobalt, with/or without promotor salts, in the synthesis of hydrocarbons from the reaction of carbon monoxide and hydrogen at substantially atmospheric pressure. Similarly International patent No WO 02/089978 to X. D. Hu, which is hereby incorporated in its entirety by reference, describes an improved ruthenium-promoted cobalt catalyst on a support, such as alumina, silica, titania, zinc-oxide, clay, zeolite and/or combinations thereof, prepared out of an aqueous solution of cobalt- with or without promotor salts, in the synthesis of hydrocarbons from the reaction of carbon monoxide and hydrogen. Cobalt based Fischer-Tropsch catalysts are discussed in “Design, synthesis and use of cobalt-based Fischer-Tropsch catalysts”, Applied Catalysis A.: General 161 (1977) 59-78, by E. Igelsia; “Practical and Theoretical Aspects of the Catalytic Fischer-Tropsch Process,” Applied Catalysis A: General 138 (1996) 319-344 by M. E. Dry, all incorporated by reference herein.

As known to the art, both the composition and the physical characteristics of the Fischer-Tropsch catalyst particles affect the catalyst activity of the catalyst. Because the hydrogen gas and carbon monoxide must make physical contact with the Group VIII metal for the conversion to occur, catalyst particles with uniform metal distribution, homogeneous metal loading and high surface areas have higher activity rates in commercial scale reactors than particles with the metal localized in lumps on the surface. Thus, it would be beneficial to have a cobalt-based Fischer-Tropsch catalyst that has a high surface area, a smooth, homogeneous surface morphology and a uniform distribution of metal over the catalyst surface. Because studies have shown that the metal crystallite size might affect the reactions, the active catalyst metal would preferably have a small crystallite size for high activity in the Fischer-Tropsch reactions. The utilization of nano particle cobalt crystallites is disclosed in Dunn, B. C. et al, “Silica Xerogel Supported Cobalt Metal Fischer-Tropsch Catalysts for Syngas to Diesel Range Fuel Conversion”, Energy & Fuels 2004, 18, 1519-1521 , which is hereby incorporated in its entirety by reference.

Not only are the sizes of the crystallites important in terms of the physical characteristics. It is well known to those skilled in the art that only the metallic form of the element selected from Group VIII of the Periodic Table of Elements (iron, cobalt, nickel, ruthenium, rhenium, palladium, osmium, iridium and platinum) is active in the Fischer-Tropsch hydrocarbon synthesis. As mentioned above the catalysts are prepared out of solutions of metal salts. Through calcination the catalytically active constituents are fixed on the catalyst surface in the form of metal oxides, generally through calcination at elevated temperatures in air. Being able to obtain the active metal form from the metal oxides is therefore critically important. This transformation of an inactive metal oxide form to the active metal is known as “activation” and encompasses some form of reduction of the metal oxide to the active metal. The quantity of metal oxides on the catalyst surface which can be reduced through the activation procedure is therefore important to the activity of the catalyst.

The catalytic activity of cobalt supported on a carrier has been found to be influenced by the interaction of carrier material and the size of the cobalt crystallites Jacobs, G. et al., Applied Catalysis A: General 233 (2002) 263-281, which is hereby incorporated in its entirety by reference. They observed that not only does choice of support largely determine the number of active sites stabilized after reduction, but it also strongly influences the percentage of the cobalt oxide species that can be reduced. Therefore, for a reduction temperature of 350° C., which is a typical standard reduction temperature for Cobalt Fischer-Tropsch synthesis catalysts, a tradeoff exists between the cobalt dispersion and the percentage of cobalt oxide species reduced. Supports such as SiO₂, which yield a large cluster size, offer the highest percentage reduction at 350° C., while supports like Al₂O₃, which stabilize a smaller cluster size, have significant support interactions which impede the reduction. That is, a Fischer-Tropsch reduction at 350° C. for 10 hours resulting in a significant fraction of the cobalt oxide species interacting with the support and remaining in a non-reduced state.

In order to gain better access to the active sites, noble metal promoters are often employed. These noble metal promoters, such as platinum (Pt) or ruthenium (Ru), reduce at a lower temperature than the cobalt oxides, and they, in turn, catalyze cobalt reduction, presumably by hydrogen spillover from the promoter surface. Thus, addition of small amounts of noble metal shifts the reduction temperature of cobalt oxides and cobalt species interacting with the support to lower temperatures.

As ruthenium is expensive, many patents indicate that it is preferred to employ ruthenium in the minimum amount necessary to achieve the desired result. Moreover, not only the added expense of the promoter needs consideration, it is also important to determine the appropriate loading of promoter to maximize the availability of active cobalt surface sites on the carrier for participation in the reaction, after catalyst activation. Attempts have been made to utilize unpromoted cobalt catalysts for the synthesis of hydrocarbons from synthesis gas. However, unpromoted cobalt often has poor selectivity and requires high metal loadings to provide desirable activity. Kobylinski describes, for this purpose, the use of a cobalt catalyst on a support with up to 30 weight percent cobalt loading. Similarly International patent No WO 02/089978 to X. D. Hu, which is hereby incorporated by reference, describes an improved supported cobalt catalyst, having up to about 60 weight% cobalt loading in order to compensate for the absence of (ruthenium) promoter.

Attempts have also been made to utilize different promoters for cobalt catalysts for the synthesis of hydrocarbons from synthesis gas. For a more extensive discussion of cobalt catalysts and promoters, see U.S. Pat. No. 5,248,701, issued to Soled et al, hereby incorporated in its entirety by reference. However, it has been found that different promoters have different side reactions and selectively produce hydrocarbons, especially olefins. Ruthenium in low concentration remains an attractive promoter as it is not only a promoter for the activation, but also a Fischer-Tropsch catalyst and, hence, combines the function of promoter and catalyst.

Not withstanding the improvement offered by the use of promoters, the activation procedure still typically takes place in a certain temperature interval/range and is successfully completed at the high end of this range, at temperatures well above the normal Fischer-Tropsch operating range of 185-250° C., and typically at 350° C. Kobylinski claims “The process of claim 1 wherein said activation is conducted at a temperature in the range of between about 250° C. and about 400° C.” Jacobs, referenced above, and others use the standard temperature of 350° C. Bezemer, G. L. et al describe the activation at 350° C. despite the use of nano crystals and a manganese promoter (Bezemer, G. L. et al, “Cobalt on carbon nanofiber catalysts: auspicious system for study of manganese promotion in Fischer-Tropsch catalysis”, Chem. Commun., 2005, 731-733, which is herby incorporated in its entirety by reference.

The activation temperatures described in patents and literature are substantially different than the normal operating temperature under which low temperature Fischer-Tropsch operation takes place, i.e. 185-250° C. This has particular implications for in-situ activation in multi-tubular reactors. Here, special design measures need to be taken to accommodate this activation procedure at higher than Fischer-Tropsch operating temperature. For example, whereas the multi-tubular reactors are normally controlled to a maximum operating temperature of 250° C., by boiling water/steam generation, and whereas the saturated steam pressure corresponding to 250° C. is about 560 psi, the corresponding steam pressure at 350° C. is over 2000 psi. In order for such reactors to accommodate in-situ activation at the standard activation conditions of 350° C., additional pressure allowances have to be made, making the multi-tubular reactors and their associated systems extremely expensive. Alternatively the catalyst can be activated ex-situ. For example, some catalyst manufacturers offer this feature against fees. Additionally the (activated) catalyst needs transfer between the activation facility and the Fischer-Tropsch reactor at the operating site, which entrails the danger of renewed contact with air, hence (partial) re-oxidation/deactivation and handling of a highly active material. In such cases additional operating costs are incurred.

### SUMMARY OF THE INVENTION

The present invention provides a process for the conversion of synthesis gas into liquid hydrocarbons (e.g. diesel, naphtha, distillates, etc.) wherein a supported, promoted cobalt catalyst is activated in situ in the Fischer-Tropsch process reactor and successfully completed at temperatures well below 350° C., allowing the use of less expensive components of equipment in plants utilized in the process. This significantly reduces the investment cost per barrel of product and/or lowers operating costs, while maintaining efficiency in the conversion process and thereby allows synthesis gas conversions via the Fischer-Tropsch process in applications that otherwise would not be commercially viable.

The low temperature activation procedure of the present invention allows not only for activation of the catalyst in-situ in a fixed tube “low design temperature” reactor. It also allows for regeneration in this reactor multiple times during the active economic life of the catalyst, without having the inconvenience and production loss coupled with unloading, regenerating ex-situ and reloading the catalyst every time. The lower temperature in-situ regeneration is even more beneficial as it has been shown to generate an improved activity of promoted, supported cobalt catalysts, wherein promoters, such as ruthenium and lanthanium have been previously added by re-dispersion of the cobalt crystallities. Activities improved by up to 40% at conditions in the range of the Fischer-Tropsch operating temperatures have been obtained.

The invention further comprises a method for activating a catalyst for the conversion of a synthesis gas comprising carbon monoxide and hydrocarbon into liquid hydrocarbon products; the method consisting of: depositing a catalyst precursor selected from oxidized cobalt and oxidized nickel on a refractory metal oxide support to distribute the catalyst precursor on the refractory metal oxide support to form a supported catalyst; and, activating the supported catalyst by contacting the supported catalyst with hydrogen at a space velocity from about 100 to about 3000 liters or more of gas per hour per liter of supported catalyst precursor, preferably about 600 to about 800 liters-per-hour per liter of catalyst at a temperature from about 100° C. up to about 300° C., and preferably below 250° C.

The invention also comprises a method for regenerating a reduced activity catalyst for the conversion of a synthesis gas comprising carbon monoxide and hydrogen into liquid hydrocarbon products; the catalyst containing a catalytic metal selected from the group consisting of cobalt and nickel supported on at least one refractory metal oxide support selected from the group consisting of alumina, silica, titanium oxide and carbon; the method consisting essentially of: contacting the reduced activity catalyst with hydrogen gas at a temperature from about 100 to about 275° C., and preferably below 250° C., according to the method described below whereby the main function of the hydrogen-activation in this step is to remove the remaining hydrocarbons and/or coke; oxidizing the reduced activity catalyst by contacting the reduced activity catalyst with an oxygen-containing gas, such as oxygen, air, oxygen-enriched air or the like, preferably at about 0.5% vol. oxygen in the gas for a time span of 12 hours during which the temperature is increased from about 100 to about 275° C., and preferably below about 150° C., to produce an oxidized catalyst; and, contacting the oxidized catalyst with hydrogen gas at a temperature from about 100 to about 275° C., and preferably below about 250° C., according to the method described herein, to produce an activated regenerated catalyst.

The invention further includes a method for the conversion of a synthesis gas comprising carbon monoxide and hydrogen into liquid hydrocarbon products by contacting the synthesis gas at a temperature from about 100° C. to about 275° C., and preferably below 250° C., with an activated catalyst consisting essentially of a catalytic metal selected from the group consisting of cobalt and nickel supported on at least one refractory metal oxide selected from the group consisting of alumina, silica, titanium oxide and carbon; the method consisting essentially of: depositing a catalyst precursor selected from the group consisting of oxidized cobalt and oxidized nickel supported on the refractory metal oxide support, adding a selected quantity of a promoter; activating the supported catalyst precursor by contacting the supported catalyst precursor with hydrogen at a temperature from about 100° C. up to about 300° C., and preferably below 250° C., and at a space velocity from about 100 to about 3000 liters or more of gas per hour per liter of supported catalyst precursor, preferably about 600 to about 800 liters-per-hour per liter of supported catalyst precursor to produce the activated catalyst; and, contacting the synthesis gas with the activated catalyst at conversion conditions to produce the liquid hydrocarbon products.

### DESCRIPTION OF PREFERRED EMBODIMENTS

According to the present invention, a method is provided for activating a catalyst for use in the conversion of a synthesis gas comprising carbon monoxide and hydrogen into liquid hydrocarbon products. The catalyst desirably consists of one or more refractory metal oxides selected from a group consisting of alumina, silica, titanium oxide and carbon with a promoter, an oxidized cobalt or an oxidized nickel or both being deposited on the refractory metal oxide to evenly distribute the catalyst precursor materials on the refractory metal oxide. The supported catalyst precursor is then subjected to activation by contacting the supported catalyst with hydrogen at a temperature from about 100° C. up to about 300° C. A preferred range is from about 100° C. to about 275° C., and preferably below 250° C.

Desirably the activation is accomplished by contacting the supported catalyst precursors in a tubular reactor adapted for the Fischer-Tropsch process. These reactors are typically designed for the conduct of the Fischer-Tropsch reaction which is normally done at temperatures from about 185° C. to about 250° C. These tubular reactors are typically water-cooled and at temperatures above 275° C. the pressure requirement for the reactor become prohibitively expensive. More specifically at 250° C., the stream pressure is about 560 pounds per square inch (psi) whereas at 350° C. the stream pressure is over 2000 psi. This greatly increases the vessel cost.

Previously, catalysts have been activated in different, smaller and dedicated reactors (ex-situ) by contacting with hydrogen at temperatures of 350° C. or higher. These higher temperature activating procedures produce an active catalyst which then must be transferred to the reaction zone which may be a tubular reactor without contacting air. Contact with air reoxidizes the active cobalt or nickel sites, thereby rendering the catalyst ineffective.

According to the present invention, the catalyst may be and preferably is activated in situ in tubular Fischer-Tropsch reactors by contacting the catalyst with hydrogen at a temperature from about 100° C. up to 300° C. and preferably from about 100° C. up to 275° C., and preferably below 250° C., with the hydrogen being passed through the catalyst at a space velocity of about 100 to about 3000 liters or more of gas per hour per liter of supported catalyst precursor, preferably about 600 to about 800 liters-per-hour per liter of catalyst. The high space velocity is beneficial in removing water produced by the activation quickly from the vicinity of the activated catalyst so that the water has little opportunity to react with the active catalyst sites and re-oxidize the active catalyst sites. Desirably the activation is conducted while heating the supported catalyst at a rate from about 0.1° C. to about 2° C. per minute. Of the catalytic metals mentioned, cobalt is preferred although combinations of cobalt with nickel may be used and the catalyst may include a promotor selected from commonly used promoters, such as platinum, ruthenium, rhenium, lanthanum and manganese and the like.

While titanium oxide and carbon may be used as the refractory oxide support, alumina and silica are preferred as the refractory oxide support. Of these, alumina is preferred as the refractory oxide support, although silica is also considered suitable. Mixtures of the refractory oxide materials with each other or minor quantities of other refractory materials may be also be suitable. These two refractory metal oxide supports are preferred because of their greater resistance to water. In other words, water is formed during the activation procedure and at the higher hydrogen space velocity is more quickly removed from the reaction zone and is less active with the treated catalyst and with the refractory metal oxide supports. Because of their greater resistance to water and the increased likelihood of water presence at the lower temperatures of the present invention, these two refractory metal oxides, alumina and silica, are preferred.

As indicated, the temperature for the activation process according to the present invention is considerably lower than the temperature typically used. The activation is at a temperature such that it may be accomplished in situ in the vessel subsequently or previously used for the Fischer-Tropsch reaction. This eliminates the requirement to move the activated catalyst without contact with air from an activation site outside the Fischer-Tropsch reactor into the reactor vessel tubes without contact with air.

Typically the catalyst may contain variable amounts of catalytic material. Typically the catalyst contains from about 10 to about 60 weight percent cobalt and preferably from about 15 to about 25 weight percent cobalt based upon the weight of the catalyst. When nickel is used, the nickel is desirably used in amount from about 10 to about 60 weight percent and preferably from about 20 to about 40 weight percent. The cobalt and nickel may be mixed in any portions desired in the catalyst.

The hydrogen may be supplied as pure hydrogen or hydrogen mixed with nitrogen or the like.

The catalyst may also include a promoter such as platinum, ruthenium, rhenium, lanthanum and manganese or the like. When the promoter is present, it is typically added in amounts from about 0.05 to about 0.5 and preferably from about 0.1 to about 0.2 weight percent based upon the weight of the catalyst. For instance with ruthenium, the carrier is typically added in an amount equal to from about 0.05 to about 0.50 weight percent based upon the weight of the catalyst.

### EXAMPLE 1

In order to better understand the advantages of the present invention in the process of conversion of syngas via the Fischer-Tropsch reaction, using a catalyst activated at low temperature, the following examples are set forth.

Commercial mixture of Sasol and UOP gamma alumina was mixed with water and citric acid to a paste, extruded to form 1.0 mm extrudates and calcined at 600° C. to form a base catalyst carrier. About 385 ml of an aqueous cobalt/ruthenium stock solution is prepared by dissolving about 306.15 g of cobalt nitrate hexahydrate and 1.89 grams of ruthenium nitrosyl nitrate in deionized water. The solution is then poured over about 400 g of the base carrier at ambient conditions in a container. A lid is placed on the container and the container is agitated by hand for about 5 minutes or until the aluminum oxide carrier is uniformly wetted. This material is dried at about 80° C. for about 10 hours with an air flow of about 1.7 standard cubic feet per hour (SCFH), and is then calcined at about 250° C. for about 4 hours with an air flow of about 10.2 SCFH sufficient to decompose the metal salts and fix the metals. The alumina carrier is intended to contain in the reduced state 20% wt. cobalt (calculated as metal) and 0.15% ruthenium (calculated as metal). Sixty grams of the catalyst was loaded in a tubular reactor, capable of activating catalysts in two different zones in two different temperature regimes, an upper zone with low temperature activation and a lower zone with high temperature activation. The catalyst in this special tubular reactor was activated according to the following procedure:

Step 1: Reduction
1. Flush with nitrogen.
2. Pressurize with nitrogen to low pressure (max 50 psia), N₂ flow at a gas hourly space velocity (GHSV) of 600-800 N liter/liter/hour.
3. Increase ambient temperature to 100° C. and hold at 100° C. for 1 hour
4. Increase temperature to 180° C.
5. At this point the upper part of the reactor will be maintained and further activated at 180° C., while the lower part will see a standard reduction temperature of up to 350° C.
6. Introduce hydrogen diluted with nitrogen and increase the hydrogen content with nitrogen and from zero to 50% vol., in increments of 10% vol. per hour.
7. Increase the hydrogen content simultaneously to 75% vol., while stepping up the temperature of the lower part of the reactor to 350° C.
8. Hold for 12 hours at 350° C. and 75% vol. hydrogen.
9. Switch back to nitrogen and cool down to 180° C.
10. Adjust nitrogen flow rate to reflect vgas=0.25 m/s (0.8 ft/sec).

Step 2: Conditioning under carbon monoxide
1. Pressurize to 590 psi.
2. Carefully introduce the first carbon monoxide targeting 2.5 vol. %.
3. After 30 minutes increase the carbon monoxide to 5% vol.
4. Increase the carbon monoxide two more times, 30 minutes apart to 10% vol.
5. Adjust total gas flow to reflect vgas=0.25 m/s (0.8 ft/sec).

Step 3: Reaction
1. Slowly introduce hydrogen at 180° C. (steam side) and 2.5% per half hour to 11% vol. target.
2. Adjust gas flow to reflect vgas=0.25 m/s (0.8 ft/sec).

Both the upper part of the reactor and the lower part of the reactor see the same flow rates and conditions except that the lower part of the reactor is heated to 350° C. by contrast to the upper portion which is only heated to 180° C.

Following the introduction of a mixture of hydrogen and carbon monoxide to the catalyst at 220° C. it was evident that the lower part of the catalyst bed had been activated and was converting at 55% carbon monoxide conversion.

In order to test the catalyst activated at 180° C. the experimental conditions (220° C., 590 psia, syngas flow at vgas=0.25 m/s) were kept constant over the time frame of 10 hours, after which the low temperature procedure had soaked in. The catalyst of the top part of the reactor was tested on conversion. It was found that the top part of the catalyst bed, which had only seen the low temperature—180° C. activation, was now converting on a par with the catalyst, activated at 350° C. (54% conversion vs. 55% conversion at 220° C. Fischer-Tropsch reaction temperature see Table 1)

### EXAMPLE 2

Two-hundred fifty grams of the same promoted cobalt-ruthenium catalyst prepared as described in Example 1 was ground and sieved to 20-40 mesh granules. Forty grams of this sieved catalyst fraction was loaded in a tubular reactor and activated according to the procedure of example 1, with the exception that the activation temperature of the entire bed was raised to a maximum of 230° C. Twenty grams of the catalyst was transferred under nitrogen to a CSTR (continuously stirred tank reactor) to be used in a slurry Fischer-Tropsch reactor. Prior to introduction of synthesis gas, the catalyst was once more reduced in situ (polished up) by feeding hydrogen/nitrogen gas into the reactor at a temperature of 230° C., a gas flow of 100 liters-per-hour, under a pressure of 15 psig, at a hydrogen concentration of 0-100% mol for 10 hours. The system was purged with nitrogen and then a Fischer-Tropsch reaction carried out using a synthesis gas feed of a 2:1 volume ratio of hydrogen to carbon monoxide, the reaction conditions in the CSTR being adjusted to a temperature of 180° C., 200° C. and 220° C. as shown below, a pressure of 500 psig and a space velocity of 1.5 Normal liters/gram dry catalyst/hour. The reaction was carried out in solvent. The effluent gas from the reactor was monitored by an HP-5840A Refinery Gas Analyzer to determine the degree of Cobalt conversion and the nature of the hydrocarbon products. The results, given in Table 1 below, show that at 220° C. Fischer-Tropsch operating temperature, this low temperature activated catalyst performs satisfactorily. Considering the operating conditions, the C₅⁺ make is comparable with rates obtained in the fixed bed reactor of Example 1.

TABLE 1

| | Example 1 | | | |
|---|---|---|---|---|
| | | C5+ make gram HC/gr | Example 2 | |
| | CO conversion @ 220° C. | cat/hr @ 220° C. | | C5+ make gram HC/gr |
| | (% mol) | (calculated) | CO conversion (% mol) | cat/hr (calculated) |
| Hour 1 (top) | 10 | 0.0225 | | |
| Hour 1 (bottom) | 55 | 0.1204 | | |
| Hour 12 (top) | 54 | 0.1180 | | |
| Hour 12 | 55 | 0.1204 | | |
| (bottom) | | | | |
| *T = 180° C. | | | 7.3 | 0.0199 |
| *T = 200° C. | | | 31.5 | 0.0802 |
| *T = 220° C. | | | 44.0 | 0.1107 |

*Fischer-Tropsch operating temperature.

While the present invention has been described by reference to certain of its preferred embodiments, it is pointed out that the embodiments described are illustrative rather than limiting in nature and that many variations and modifications are possible within the scope of the present invention. Many such variations and modifications may be considered obvious and desirable by those skilled in the art based upon a review of the foregoing description of preferred embodiments.

1. A method for activating a supported catalyst for the conversion of a synthesis gas comprising carbon monoxide and hydrocarbon into liquid hydrocarbon products; the supported catalyst being activated in situ in a Fischer-Tropsch reactor, the method consisting essentially of:
(a) depositing a catalyst oxide precursor precursor being selected from oxidized cobalt and oxidized nickel on a refractory metal oxide support to distribute the catalyst precursor on the refractory metal oxide support to form the supported catalyst; and,
(b) activating the supported catalyst by contacting the supported catalyst with a hydrogen-containing gas at a space velocity from about 100 to about 3000 Nliters-per-hour per liter of catalyst at a temperature from about 100° C. up to 300° C.

2. The method of claim 1 wherein the refractory metal oxide is selected from the group consisting of alumina, silica, titanium oxide and carbon.

3. The method of claim 1 wherein the temperature is from about 100° C. to 275° C.

4. The method of claim 1 wherein the temperature is from about 100° C. to 250° C.

5. The method of claim 1 wherein the activation is conducted while heating the supported catalyst at a rate from about 0.1° C. to about 2° C. per minute.

6. The method of claim 1 wherein the supported catalyst contains from about 10 to about 60 weight percent cobalt.

7. The method of claim 1 wherein the supported catalyst contains from about 10 to about 60 weight percent nickel.

8. The method of claim 1 wherein the supported catalyst contains both cobalt and nickel.

9. The method of claim 1 wherein the supported carrier further contains a promoter.

10. The method of claim 9 wherein the promoter comprises at least one of platinum, ruthenium, rhenium, lanthanum or manganese.

11. A method for regenerating a reduced activity catalyst for the conversion of a synthesis gas comprising carbon monoxide and hydrogen into liquid hydrocarbon products; in a Fischer-Tropsch reactor the catalyst containing a catalytic metal selected from the group consisting essential of cobalt and nickel supported on a refractory metal oxide support selected from the group consisting of alumina, silica, titanium oxide and carbon; the method consisting essentially of:
(a) contacting the reduced activity catalyst with a hydrogen-containing gas at a temperature from about 100° C. to 300° C.;
(b) oxidizing the reduced activity catalyst by contacting the reduced activity catalyst with an oxygen-containing gas at a temperature from about 100 to 275° C. to produce an oxidized catalyst; and,
(c) contacting the oxidized catalyst with a hydrogen-containing gas at a space velocity from about 100 to about 3000 N liter per hour per liter of catalyst at a temperature from about 100 to 300° C. to produce an activated regenerated catalyst.

12. The method of claim 11 wherein the temperature of the oxidized catalyst is increased by from about 0.1° C. to about 2° C. per minute during the hydrogen contacting.

13. The method of claim 11 wherein the catalyst comprises cobalt or alumina.

14. The method of claim 11 wherein the catalyst further contains a promoter.

15. The method of claim 11 wherein the promoter is selected from the group consisting of platinum, ruthenium, rhenium, lanthanum and manganese.

16. A method for the conversion of a synthesis gas comprising carbon monoxide and hydrogen to liquid hydrocarbon products by contacting the synthesis gas at a temperature from about 100° C. to 275° C. with an activated catalyst consisting essential of a catalyst metal selected from the group consisting of cobalt and nickel supported on a refractory metal oxide selected from the group consisting of alumina, silica, titanium oxide and carbon in a Fischer-Tropsch reactor; the method consisting essential of:
(a) depositing a catalyst precursor in the Fischer-Tropsch reactor selected from the group consisting of oxidized cobalt and oxidized nickel supported on the refractory metal oxide support;
(b) activating the supported catalyst precursor in the Fischer-Tropsch reactor the supported catalyst being activated by contacting the supported catalyst precursor with a hydrogen-containing gas at a temperature from about 100° C. up to about 300° C. and at a space velocity from about 100 to about 3000 Nliters per liter of supported catalyst precursor to produce the activated catalyst; and,
(c) contacting the synthesis gas with the activated catalyst at conversion conditions to produce the liquid hydrocarbon products in a fixed bed Fischer-Tropsch reactor or slurry bed Fischer-Tropsch reactor.

17. The method of claim 16 wherein the refractory metal oxide is alumina.

18. The method of claim 16 wherein the activation is conducted at an increasing temperature up to about 250° C., the temperature being increased at a rate from about 0.1° C. to about 2° C. per minute.

19. The method of claim 16 wherein the supported catalyst precursor contains a promotor.

20. The method of claim 16 wherein the promoter is selected from the group consisting of platinum, ruthenium, rhenium , lanthanum and manganese.
